(19) United States
(12) Patent Application Publication
(10) Pub. No.: US 2020/0104285 A1
(43) Pub. Date: Apr. 2, 2020

(54) SIGNATURE-HASH FOR MULTI-SEQUENCE FILES

(71) Applicant: NANTOMICS, LLC, Culver City, CA (US)

(72) Inventors: John Zachary SANBORN, Culver City, CA (US); Stephen Charles BENZ, Culver City, CA (US); Rahul PARULKAR, Culver City, CA (US)

(21) Appl. No.: 16/499,164

(22) PCT Filed: Mar. 28, 2018
(86) PCT No.: PCT/US2018/024838

§ 371 Date: Sep. 27, 2019

(51) Int. Cl. G06F 16/22; G16B 20/20; G16B 50/40; G16B 30/00

(52) U.S. Cl. CPC G06F 16/2255; G16B 30/00; G16B 50/40; G16B 20/20

(57) ABSTRACT

A unique hash representing patient omics data is constructed using results for known SNP positions and their respective allele frequencies in the patient's omics data. In most preferred aspects, the known SNP positions are selected for specific factors (e.g., ethnicity, sex, etc.) and the allele fraction is represented in values of a non-linear scale. Typically, the hash comprises a header/metadata relating to the known SNP positions and non-linear scale and further includes the actual hash string.

This application claims priority to our copending US provisional application with the Ser. No. 62/478,531, which was filed Mar. 29, 2017.

### FIELD OF THE INVENTION

The field of the invention is validation systems and methods for detection of genetic variation, especially as it relates to rapid identification and/or matching of sequence data for whole genome analysis.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Single nucleotide polymorphism (SNP) refers to the occurrence of a variant or change at a single DNA base pair position among genomes of different individuals. Notably, SNPs are relatively common in the human genome, typically at a frequency of about 10⁻³, and are often indiscriminately located in both transcriptional and regulatory/non-coding sequences. Because of their relatively high frequency and known positions, SNPs can be used in various fields and have found several applications in genome-wide association studies, population genetics, and evolution studies. However, the vast amount of information has also resulted in various challenges.

For example, where SNPs are used in genome-wide association studies, an entire genome has to be sequenced for many individuals from at least two distinct groups to obtain statistically relevant association of a marker or disease with a SNP or SNP pattern. On the other hand, where only a fraction of the genome or selected SNPs are analyzed, potential associations may be lost as the SNPs are widely distributed throughout an entire genome. In still other methods of using SNPs, polymorphisms can be targeted. However, in such case dedicated equipment (high-throughput PCR) and/or materials (SNP arrays) are generally required. In addition, once a base pair position is identified as being the locus of a SNP, such information is typically only deemed useful where a particular SNP is associated with one or more clinical features. Thus, many SNPs for which no condition or feature is known are simply deemed irrelevant and disregarded.

Agnostic use of SNPs (i.e., use of SNP without accounting for any association with a condition or disease) as a sample-specific idiosyncratic marker was recently described in WO 2016/037134. Here, a plurality of predetermined SNPs were used as identifiers using a base read with complete disregard of any clinical or physiological consequence of the read in the SNP locus. Thus, a relatively large number of SNPs provides a unique constellation of idiosyncratic markers that could be used to track the provenance of a sample. However, such systems fail to account for allelic variation of SNPs. Moreover, use of SNPs to produce a marker profile will not allow identification of relationships for a number of samples and/or sample purity/contamination of a sample.

Most commonly, the relationship for omics data for a number of samples (e.g., first, second, and subsequent biopsies) is based on patient identifiers in the data file along with other sample relevant information. Unfortunately, where a sample is mislabeled or otherwise changed, incorrect patient identifiers will make it difficult, if not impossible, to rectify such mistakes. Likewise, where one patient sample is contaminated with another patient sample or a sample of an earlier point in time, currently known data processing will typically not allow identification of such contamination. Still further, where sample matching or sample retrieval of a sample based on sequence information only is desired, currently known systems and methods will typically require full sequence comparisons and/or alignments. Viewed from a different perspective, currently known systems for sequence retrieval, identification, and/or matching rely on computationally ineffective alignments, or on header data that may be inaccurate. Known SNP analysis failed to address these issues.

Thus, even though various aspects and methods for SNPs are known in the art, there is still a need for improved systems and methods that leverage SNPs as an information source.

### SUMMARY OF THE INVENTION

The inventive subject matter is directed to various devices, systems, and methods for generating a unique signature-hash for an omics data set (typically for a SAM, Bam, or GAR file) by converting raw read allele frequencies for known SNP sites into a typically non-linear (e.g., dynamic hexadecimal) representation and storing the so obtained data as a hash string in a database. Such data structure is particularly advantageous for increasing speed and reducing computational resource demand when, for example, matching or retrieving specific omics data sets, and identifying sample contamination or sample provenance.

In one aspect of the inventive subject matter, the inventors contemplate a method of generating a signature-hash that includes a step of identifying in an omics data set a plurality of SNPs (single nucleotide polymorphisms) in respective selected locations, and a further step of determining allele frequencies for the plurality of SNPs. In another step, respective values are assigned to the plurality of SNPs based on the allele frequencies, and an output file is generated that comprises the values for the plurality of SNPs as well as metadata related to the selected locations.

Most typically, but not necessarily, the omics data set comprises raw sequence reads, and it is further contemplated that the omics data set will have a SAM format, BAM format, or GAR format. While not limiting to the inventive subject matter, it is also contemplated that the selected locations will be selected on the basis of SNP frequency, gender, ethnicity, and/or mutation type. Moreover, it is also contemplated that the values are based on a non-linear scale, and may be expressed as hexadecimal values. Most typically, the values for the plurality of SNPs are stored in a single string, and metadata (e.g., relating to scale information for the values, choice, type, location of SNPS, etc.) may be located in a separate header. In further contemplated methods, the signature-hash is associated with the omics data set.

Therefore, and viewed form a different perspective, the inventors also contemplate a method of comparing a plurality of omics data sets. In such method, a first signature-hash is obtained or generated for a first omics data set, and a second signature-hash is obtained or generated for a second omics data set. Most typically, each of the first and second signature-hashes will comprise a plurality of values that correspond to allele frequencies for a plurality of SNPs in selected locations of the second omics data sets and further comprise metadata related to the selected locations. In another step, the plurality of values for the first and second signature-hashes are then compared to determine a degree of relatedness.

Preferably, first and second omics data sets will be in a SAM format, BAM format, or GAR format, and/or the locations may be selected on the basis of SNP frequency, gender, ethnicity, and/or mutation type. As noted above, the values may be based on a non-linear scale, and/or be expressed as hexadecimal values. Most typically, the first omics data set comprises the first signature-hash, and the second omics data comprises the second signature-hash. In still further contemplated aspects, the degree of relatedness may be based on SNP frequency, gender, ethnicity, and mutation type, and it is noted that a predetermined degree of relatedness may be indicative of common provenance.

In still further contemplated aspects, the inventors also contemplate a method of identifying a single omics data set in a plurality of omics data sets having respective signature-hashes. In such method, a single signature-hash is obtained or generated that has a predetermined degree of relatedness to the single omics data set. Most typically, each of the signature-hashes comprises a plurality of values corresponding to allele frequencies for a plurality of SNPs in selected locations of an omics data set and further comprises metadata related to the selected locations. In a further step, the plurality of values are compared for the single signature-hash with values of the signature-hashes of each of the plurality of omics data sets, and in yet another step, the single omics data set is identified in the plurality of omics data sets on the basis of a degree of relatedness between the values of the single signature-hash and values of the signature-hashes of each of the plurality of omics data sets.

Among other options, the single signature-hash may be obtained or generated from an additional omics data set, and the predetermined degree is identity or similarity of at least 90% of the plurality of values. Where desired, the single omics data set may then be retrieved. Most typically, the step of comparing will use the metadata.

Moreover, in yet another aspect of the inventive subject matter, the inventors also contemplate a method of identifying source contamination in an omics file. Such method will preferably comprise a step of providing a plurality of omics data sets having respective signature-hashes, wherein each of the signature-hashes comprises a plurality of values corresponding to allele frequencies for a plurality of SNPs in selected locations of an omics data set and further comprises metadata related to the selected locations. In a further step, at least some of the plurality of values of one of the omics data set are then identified in another omics data set.

Most typically, at least two of the plurality of omics data sets will be from the same patient and are representative of at least two distinct points in time. Additionally, it is contemplated that the selected locations are based on for at least one of SNP frequency, gender, ethnicity, and mutation type, while the step of identifying comprises a step of subtraction of corresponding values between at least two omics data sets. Where desired, such methods may further comprise a step of identifying metadata in the one of the omics data sets.

Various objects, features, aspects and advantages of the inventive subject matter will become more apparent from the following detailed description of preferred embodiments, along with the accompanying drawing FIGURES in which like numerals represent like components.

### BRIEF DESCRIPTION OF THE DRAWING

FIG. 1 is an exemplary signature-hash for a BAM file according to the inventive subject matter.

### DETAILED DESCRIPTION

The inventors have discovered that various otherwise computationally demanding processes for analysis of omics data sets (e.g., determination of provenance or contamination of a sample, sample retrieval or comparisons, etc.) can be performed in a conceptually simple and efficient manner in which allele frequencies of a plurality of SNPs are used as ‘weighted’ proxy markers for a specific sample. Advantageously, such information can be expressed as a hash that is associated with the omics data (the terms ‘signature-hash’ and ‘hash’ are used interchangeably herein. Viewed form a different perspective, it should be noted that the systems and methods contemplated herein not only make use of high entropy markers among various related sequences to so provide a static picture (i.e., SNP present or not present), but also employ allele frequency to so allow for a weighted analysis that adds higher information content (i.e., the SNP is present at a specific fraction), that also allows identification of two or more distinct patterns present in the same data set.

Indeed, it should be recognized that contemplated systems and methods now allow for identification, matching, and/or comparison of partial (e.g., whole exome, transcriptome, or selected genes) or even whole genome omics data in a manner that is independent of patient or sample identifiers but that is based on the entirety of the analyzed sequence information. Thus, instead of requiring a comprehensive sequence analysis on a nucleotide-by-nucleotide basis for the entirety of two or more sequences, simplified (but equally informative) analysis can be performed using the hash that is associated with the respective omics data. Moreover, it should be recognized that using the hash that is associated with the omics data, similarity searches with predefined inclusion/exclusion criteria can be performed without the need to perform analyses on a nucleotide-by-nucleotide basis for the entirety of the sequences under investigation. Thus, the computationally very small (typically only a few kilobytes or even less) and simple hash contemplated herein can be used as a sample-specific proxy for a very large (typically several hundred gigabytes) and complex whole genome data file (e.g., BAM, SAM, or GAR file with a very large number of individual sequence reads).

For example, in one typical aspect of the inventive subject matter, a unique hash for a whole genome sequence of a patient sample is constructed using the omics data in the whole (or partial) genome sequence file. For example, sequence information of all reads in a BAM or SAM file may be used to obtain base call and allele frequency data for a particular position in the genome. Especially preferred positions in the genome are those known to be a locus for a SNP. As will be readily appreciated, more than one known SNP position will be used in the methods contemplated herein to generate statistically unique and significant results. Among other options, SNP base call and allele frequency can be recorded at least 10, or at least 20, or at least 50, or at least 100, or at least 500, or at least 1,000, or at least 2,000, or at least 3,000 (or more) known SNP positions.

Moreover, in most preferred aspects, the known SNP positions are selected for one or more specific factors (e.g., ethnicity, gender, genealogy, etc.), and/or the allele fraction is represented in values of a non-linear scale to allow for an increased resolution to lower allele counts near zero and less resolution once near higher allele counts. Such weighted value system is especially useful to identify sources of contamination as, for example, the major genotype from patient A can be seen at low allele frequencies in the omics data of patient B. Still further, it is generally preferred that the actual SNP positions and details (e.g., location, relevance, etc.) are encoded in a signature string that is typically delimited from the allele frequencies (e.g., by special characters), which will further advantageously allow for the determination whether or not two signatures are the same “version”. Storing such a small string beneficially allows for rapid matching/comparisons in a relational database.

With respect to the omics data sets suitable for use herein, it is generally contemplated that all omics data sets are deemed appropriate so long as they contain sufficient information to allow determination of a SNP location and associated base call(s) and contain sufficient information to allow determination of an allele frequency at a SNP location. Therefore, it should be appreciated that suitable omics data sets will include BAM files, SAM files, GAR files, etc. Alternatively, suitable omics data sets may also be based on VCF files, or previous sequence analyses that provide a plurality of SNP positions and allele frequency information for the SNP positions. Therefore, and viewed from a different perspective, contemplated omics data sets will include multiple reads, typically at a coverage depth of at least 10×, or at least 20×, or at least 50×, or at least 100×, where the multiple reads extend over at least 10%, more typically at least 20%, even more typically at least 50%, and most typically at least 75% (e.g., 90-100%) of the entire genome of a subject. Such reads will typically be aligned to conform to a particular file format, or may be unaligned and later processed to locate the SNP positions. Viewed from another perspective, it should be appreciated that the starting material for determination of the SNPs is in most cases not a patient tissue, but an already established sequence record (e.g., SAM, BAM, GAR, FASTA, FASTQ, or VCF file) from a nucleic acid sequence determination such as from whole genome sequencing, exome sequencing, RNA sequencing, etc. Consequently, the patient sample/starting material can be represented by a digital file storing multiple sequences stored according to one or more digital formats.

Where raw data files are provided (e.g., from a sequencer or sequencing facility), it should be appreciated that these data may be processed in a variety of manners to obtain an omics data set from which determination of a SNP position and associated base call(s) and allele frequency at the SNP position. Thus, raw sequence reads may be processed to align to a reference genome to so form a SAM or BAM file, and the SAM or BAM files may then be analyzed using software tools known in the art (e.g., BAMBAM as described in U.S. Pat. Nos. 9,646,134, 9,652,587, 9,721,062, 9,824,181; or variant callers such as MuTect (Nat Biotechnol. 2013 March; 31(3):213-9), HaploTypeCaller, and Strelka2 (Bioinformatics, Volume 28, Issue 14, 15 Jul. 2012, Pages 1811-1817)).

With respect to SNPs it is contemplated that all known SNPs are deemed appropriate for use herein, and especially preferred SNPs include common (rather than rare) SNPs. For example, there are numerous publicly and/or commercially available SNP databases known in the art and all of those can be used to identify and/or select SNPs for the practice of the inventive concept presented herein. For example, suitable SNP databases include dbSNP (NCBI), dbSNP-polymorphism repository (NIH), GeneSNPs (Public Internet Resource, University of Utah Genome Center team), Leelab SNP Database (UCLA Center for Bioinformatics), Single Nucleotide Polymorphisms in the Human Genome—SNP Database (Pui-Yan Kwok Washington Univ. St. Louis), The Human SNP database (Whitehead Institute/MIT Center for Genome Research), etc. Further suitable sources of SNPs include all published materials that link one or more SNPs to a condition or disease (e.g., disease or trait association studies), as well as prior sequencing data for the same patient (e.g., to identify newly arisen SNPs) as described below.

However, it is generally preferred that the SNPs are selected according to one or more further criteria that may be relevant to the characterization and/or history of an omics data set, and especially contemplated criteria include SNP frequency, gender, ethnicity, and mutation type. For example, SNPs are typically preferred where the SNP is relatively common (e.g., SNP occurs at least in 10%, or at least in 20%, or at least in 30%, or at least in 50%, or at least in 70% of the population), or where the SNP is associated with male or female gender. Likewise, it is typically preferred that the SNP may also be specific to an ethnic population (e.g., specific for AMR, FIN, EAS, SAS, AFR, etc.). On the other hand, SNPs may also be associated with a particular type of mutation (e.g., UV exposure, smoke associated damage). Moreover, SNPs may also be selected on the basis of a particular trait or condition or disease that is associated with the SNP. Of course, it should be recognized that the SNPs in the hash may also be based on multiple different parameters as discussed above. In still further and less contemplated aspects, the SNPs can also represent neoepitopes of a single sample (i.e., representing a base change resulting in a non-sense or mis-sense mutation), and so may be useful to quickly identify or retrieve omics data sets from the same patient or tumor. In such case, such hash may be useful to identify a shift in the clonal composition and/or mutational pattern.

Most typically, contemplated hashes will include values for at least 10, or at least 30, or at least 50, or at least 100, or at least 200, or at least 500, or at least 1,000 (and even more) SNPs, which may be evenly or randomly distributed throughout the genome, or which may have predetermined selected locations. Alternatively, SNPs may also be limited to specific genes, chromosomes, and/or to the exome, transcriptome, or other sub-genomic area. However, it is generally preferred that the SNPs will be sampled throughout the entire genome.

With respect to allele frequency determination of the SNP, it should be appreciated that all manners of determination are deemed suitable for use herein. For example, SNP allele frequency may be determined based on synchronous incremental alignment of multiple BAM files as described above, or from a single BAM file by analyzing the known position of the SNP. Most typically (but not necessarily), allele frequency will be expressed as a percentage value or a percentage range. Thus, it should be recognized that the value assigned to the determined allele frequency may also vary considerably, and all numeric and symbolic values are deemed suitable for use herein. However, in especially preferred aspects, values will be based on allele frequency ranges, and each range may then be assigned a particular numerical or symbolic value. The allele frequency values may be recorded in a linear scale or in a non-linear scale, and it is generally preferred that the allele frequency values will be represented on a non-linear scale with a higher resolution at lower allele frequencies.

For example, where the value range is expressed in a hexadecimal system, the allele frequency range of 0-1% could be expressed as ‘1’, the allele frequency range of 1-3% could be expressed as ‘2’, the allele frequency range of 3-5% could be expressed as ‘3’, the allele frequency range of 5-10% could be expressed as ‘4’, which will advantageously allow for the construction of a non-linear scale (i.e., more total values used for smaller range of allele frequencies, such as ten values used for a range of allele frequencies between 0 and 15%, and six values for a range of allele frequencies between 16 and 100%), which in turn will increase the resolution of downstream analytic capability for a desired allele frequency range. Thus, it should be appreciated that a value representation of allele frequencies not only allows for the distinction of two different samples even where the same number of SNPs are surveyed, but also allows generating a dynamic range (i.e., asymmetric distribution of values as discussed above) for allele frequencies. Moreover, it should be noted that different SNPs may have different value representation of allele frequencies such that allele frequencies for some SNPs may be represented on a linear scale, while other SNPs may be represented on a non-linear scale.

In addition, contemplated hashes will typically also include metadata associated with the value string, wherein the metadata will preferably include information about the type of SNPs selected, number of SNP selected, and scale information (e.g., how values are assigned to a particular numerical or symbolic value, whether or not the scale is linear or non-linear, etc.). Such information may be further encoded, or be provided as reference information to another file containing such information.

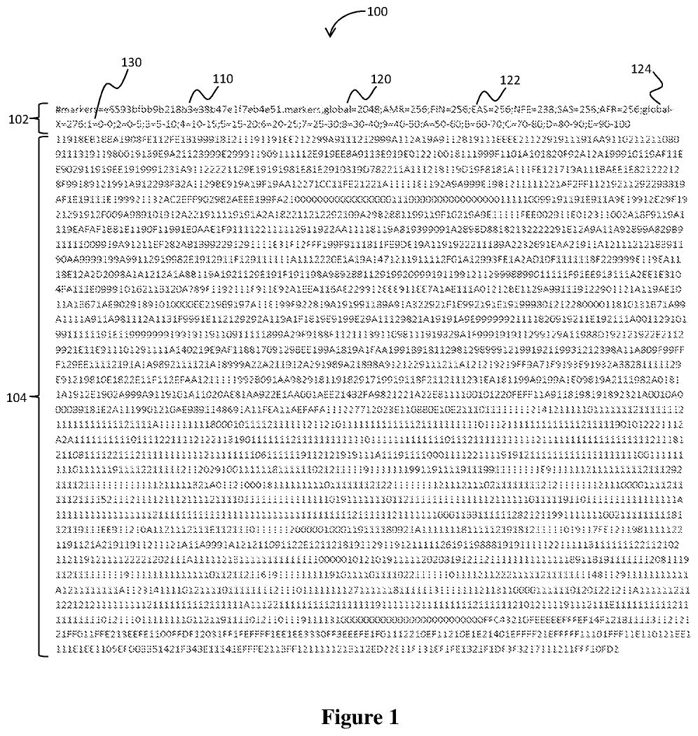

FIG. 1 depicts an exemplary hash 100 for a whole genome sequence BAM file that includes a header section 102 that is followed by values 104 for the SNPs. More specifically, the header 102 includes location reference/file name 110 of a file containing the information about the location of the SNPs, followed by specific indicators of selected SNP groups for all SNPs. Here, the exemplary group 120 denotes that 2048 SNPs were selected throughout the entire autosomal genome, while exemplary group 122 EAS (East Asian) denotes the number of ethnic specific SNPs, along with further ethnic groups such as AMR, FIN, SAS, etc., and gender specific group 124 is limited to SNPs on the X chromosome as shown in FIG. 1. As can also be seen from scale information 130, allele frequencies are expressed as ranges that have respective hexadecimal values on a non-linear scale. Of course, it should be appreciated that the hash and header may vary considerably, depending on the type and number of SNPs, as well as scaling information, and other factors. For example, the hash may further include additional information such as a patient identifier, patient/treatment history, reference to related omics data and/or files, identify and/or similarity scores to other records in a database storing multiple omics and/or hash files, etc.

It should be appreciated that contemplated hash methods are entirely independent of knowledge of SNP association with any disease or disorder, and that the hash is built only on the presence and allele frequency of the specific base call at the SNP. Thus, SNPs as used herein are also independent of the gain or loss of function. While such use advantageously allows for fast identification, processing, comparison, and analysis, contemplated methods need not be limited to known and common SNPs. Indeed, using contemplated systems and methods, it should be recognized that tumor and patient specific mutations may be followed over the course of treatment and location and allele frequencies recorded to identify clonal drift, appearance, or clearance of a tumor cell population or metastasis that is characterized by a specific SNP pattern and allele frequency. Viewed form a different perspective, tumor and patient specific mutations may be treated as the SNPs described above.

As will be readily appreciated, tumor and patient specific mutations may be identified by first comparing tumor versus normal genomic sequences to so obtain the patient and tumor specific mutation (tumor SNP). Any subsequent sequencing of a tumor or metastasis will result in a second omics data set that can then be compared against the tumor and/or normal genomic sequences that were earlier obtained to so generate secondary tumor/metastasis SNP information. It should be noted that use of the allele frequency in such methods beneficially allows tracking of SNPs that are genuine to a subpopulation/subclone of the tumor.

Moreover, it should be recognized that contemplated hash methods may be applied beyond SNPs to known mutations, or even the (dys)function of one or more known cancer-associated genes (i.e., genes that are mutated or abnormally expressed in cancer across a patient population diagnosed with the same cancer). For example, in yet another aspect of the inventive subject matter, the inventors also contemplate that somatic signature-hashes can be created from an omics record that describe/summarize somatic alterations to one or more cancer genes. For example, one contemplated exemplary encoding scheme is shown in Table 1:

TABLE 1

| Observation | Value |
|---|---|
| No Alteration | 0 |
| Copy Loss | 1 |
| Copy Gain | 2 |
| Involved in Fusion | 3 |
| Missense SNV/In-Frame Indel | 4 |
| Premature Stop | 5 |
| Copy Loss + Fusion | 6 |
| Copy Loss + Missense SNV/In-Frame Indel | 7 |
| Copy Loss + Premature Stop | 8 |
| Copy Gain + Fusion | 9 |
| Copy Gain + Missense SNV/In-Frame Indel | A |
| Copy Gain + Premature Stop | B |
| Fusion + Missense SNV/In-Frame Indel | C |
| Fusion + Premature Stop | D |
| Missense SNV/In-Frame Indel + Premature Stop | E |
| Not Analyzed | F |

In this context, and similar to the discussion above, it should be appreciated that the encoding scheme is not necessarily limited to a hexadecimal notation, and that all other notations are also deemed suitable for use herein. Moreover, a second digit may be used to encode the allele frequency of the mutation as applicable and as described above. Encoding may be performed genome-wide (e.g., covering at least 60%, or at least 75%, or at least 90%, or all of the genome), or may cover the exome only, and/or may cover the transcriptome. Moreover, it should be appreciated that the encoding may be performed on only selected genes, for example, on known cancer driver genes, mutated genes known from prior analyses of the same patient, etc. Among other scenarios, a typical encoding may thus make reference to a gene and its associated mutational status. Status will typically be based on VCF level results and/or other variant filters, but may also include customized parameters, possibly even with further reference to one or more patient specific parameters (e.g., prior treatment outcome, anticipated treatment, etc.). Thus, exemplary results may be presented as gene name and associated encoding: ATM=8, CDKN2A=0, KRAS=4 . . . PIK3CA=4, ERBB2=2, TP53=5->signature=“804 . . . 425”.

It should be particularly appreciated that contemplated somatic signatures of a panel of, for example, 500 cancer genes would result in a file of just 500 bytes. Likewise, an entire transcriptome could be encoded in approximately 25 kb. As should be readily recognized, such encoding will enable retaining even very large numbers of samples within memory for one or more downstream analyses. Still further, it should be noted that contemplated somatic signatures may computationally group similar cancers based on similar patterns of alterations, and as such quickly allow identification of potential “patients like me” from a large database of samples that could then trigger further analyses using the complete VCF datasets and/or patient EMR records, integrate with patient outcomes, to do “on-the-fly” outcome analysis with features derived from the somatic signature, etc.

Thus, it should be appreciated that the hash format presented herein is particularly useful in situations where very large sets of data need to be compared, identified by identify or degree of similarity, or analyzed for contamination or clonal fractions. Indeed, rather than analyzing the entire contents of these large files, which would occupy significant memory for processing, contemplated methods use the hash information for such purpose. Moreover, by determining the degree of granularity (e.g., SNP, or patient and tumor specific mutation, or change in structure or expression of known genes), multiple omics files can be analyzed in a highly efficient manner by only processing information provided in the hash. Indeed, using the hash information allows identification of sample contamination, for example, where two samples have been processed using the same equipment. In such case, low frequencies for a specific allele pattern can be observed in a majority allele pattern. In fact, where omics files are indexed using the hash information, individual sequence files may be retrieved from a large database (e.g., on the basis of desired identity or similarity) by only using the hash information. Advantageously, such retrieval and identification will operate independently from patient identifiers. Thus, and viewed from a different perspective, the hash information may be used as a high-entropy proxy for comparing a plurality of omics data sets by simple comparison or calculation of value information from the SNPs as expressed in the hash. Likewise, contemplated methods also include those for identifying a single omics data set in a plurality of omics data sets having respective hashes by comparing the query hash value information with value information from the SNPs as expressed in the hash of the plurality of omics data sets.

Due to the value generation of the allele frequencies, it should also be appreciated that patterns of one hash may also be detected in another hash, typically by identifying at least some of the plurality of values of one of the omics data set in another omics data set. Thus, it should be recognized that the hash values may be compared for identity or similarity (e.g., difference no larger than predetermined value), and that hash values may be subtracted from each other to so obtain a similarity score. Of course, it should be appreciated that numerous other operations than subtraction of the hash values are also deemed suitable for use herein, including binning into ranges of values, adding, sorting by ascending or descending order, etc. Moreover, as the SNPs included in the hash may be selected for specific indicators (e.g., ethnicity, gender, disease type, etc.), the hash may also be used to group omics data by the particular indicators. Likewise, as specific SNPs or other point mutations also follow a particular pattern (e.g., smoking related mutations, UV irradiation associated mutations, DNA repair defect patters, etc.), the hash may also be used to group omics data by the particular pattern.

Most typically, contemplated systems and methods will be executed on one or more computers that are informationally coupled to one or more omics databases that store or have access to omics data as discussed above. A hash-generator module is then programmed to generate a hash for an omics data set, and the hash may be attached to the omics data set or stored separately. An execution module is then programmed to use one or more hashes according to a particular task (e.g., use a specific hash to retrieve an omics data record based on the hash for that sequence, or use a specific hash to identify a plurality of omics data records based on the respective hashes).

It should be noted that any language directed to a computer should be read to include any suitable combination of computing devices, including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges preferably are conducted over a packet-switched network, the Internet, LAN, WAN, VPN, or other type of packet switched network.

### EXAMPLES

A tumor sample (T1) was discovered by an independent assay as mismatching its normal counterpart (N1) from the same patient during tumor-matched normal sequence analysis. There were two other normal samples prepared in parallel with N1 (N2, N3). Using a hash signature as described above (see also FIG. 1), the % similarity, sex, and ethnicity were determined for all 6 pairings, as shown in Table 2 below. % Similarity between a given pair of samples (i, j) was calculated according to the Equation 1 for n loci sequenced by both samples. In this example, all samples were inferred to be European (=NFE (Non-Finnish European)+FIN (Finnish European)) based on the majority of population-specific loci with AF>20% belonging to the NFE or FIN populations in their hash-signatures. Furthermore, all samples were classified as female based on exhibiting fewer than 90% of X-specific loci with heterozygous AF (i.e., 25%<AF<75%) in their hash-signatures. All mismatched samples, including the original mismatched pair (T1−N1) exhibit similarity percentages below 73%. The % Similarity for one pairing (T1−N2) was calculated to be well above these mismatched samples (94.9%), thus discovering the true matched-normal sample of tumor T1.

$$\% Similarity ( i , j ) = 1.0 - \sqrt{\frac{1}{n} {\sum }_{m = 0}^{n} {( {AF}_{m}^{i} - {AF}_{m}^{j} )}^{2}} \qquad Equation 1$$

TABLE 2
Discovery of True Sample Pairing from Similarity of Hash Signatures

| Sample A | Sample B | % Similarity | Sample A Info | Sample B Info |
|---|---|---|---|---|
| T1 | N1 | 70.6% | EUR, Female | EUR, Female |
| T1 | N2 | 94.9% | EUR, Female | EUR, Female |
| T1 | N3 | 70.4% | EUR, Female | EUR, Female |
| N1 | N2 | 72.0% | EUR, Female | EUR, Female |
| N1 | N3 | 71.5% | EUR, Female | EUR, Female |
| N2 | N3 | 72.1% | EUR, Female | EUR, Female |

To expand on the above example, we searched a larger database of clinical samples (N=173) for a match of a single target sample (A, inferred to be Asian (=EAS+SAS) Male based on its hash-signature). To speed the search, we first restricted the query sample set to Male samples that also belong to the Asian population (both previously inferred from their hash-signatures), which reduced the number of query samples from 173 to 3 (>98% reduction). It should be appreciated that such a large reduction in query samples can enable sample search to occur in real-time. Amongst that query set, we then calculated % similarity scores between the target sample and the 3 query samples. The results are summarized in Table 3 below, which show the matching query sample has % Similarity=92.8% to the target sample, which is well above the 2 remaining samples.

TABLE 3
Discovery of Sample Pairing amongst “Asian Male”-Inferred
Hash Signatures

| Target | | | | |
|---|---|---|---|---|
| Sample | Query Sample | % Similarity | Target Info | Query Info |
| T1 | Q1 | 92.8% | Asian Male | Asian Male |
| T1 | Q2 | 73.6% | Asian Male | Asian Male |
| T1 | Q3 | 74.0% | Asian Male | Asian Male |

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refers to at least one of something selected from the group consisting of A, B, C . . . and N, the text should be interpreted as requiring only one element from the group, not A plus N, or B plus N, etc.

What is claimed is:

1. A method of generating a hash for an omics data set, comprising:
identifying in an omics data set a plurality of single nucleotide polymorphisms (SNPs) in respective selected locations;
determining allele frequencies for the plurality of SNPs, and assigning respective values to the plurality of SNPs based on the allele frequencies; and
generating an output file that comprises the values for the plurality of SNPs and that further comprises metadata related to the selected locations.

2. The method of claim 1 wherein the omics data set comprises raw sequence reads.

3. The method of any one of the preceding claims, wherein the omics data set has a format selected from the group of a SAM format, BAM format, and GAR format.

4. The method of any one of the preceding claims, wherein the selected locations are selected for at least one of SNP frequency, gender, ethnicity, and mutation type.

5. The method of any one of the preceding claims, wherein the values are based on a non-linear scale.

6. The method of any one of the preceding claims, wherein the values are expressed as hexadecimal values.

7. The method of any one of the preceding claims, wherein the values for the plurality of SNPs are in a single string.

8. The method of any one of the preceding claims, wherein the metadata are located in a separate header.

9. The method of any one of the preceding claims, wherein the metadata comprise scale information for the values.

10. The method of any one of the preceding claims further comprising a step of associating the signature-hash with the omics data set.

11. The method of claim 1, wherein the omics data set has a format selected from the group of a SAM format, BAM format, and GAR format.

12. The method of claim 1, wherein the selected locations are selected for at least one of SNP frequency, gender, ethnicity, and mutation type.

13. The method of claim 1, wherein the values are based on a non-linear scale.

14. The method of claim 1, wherein the values are expressed as hexadecimal values.

15. The method of claim 1, wherein the values for the plurality of SNPs are in a single string.

16. The method of claim 1, wherein the metadata are located in a separate header.

17. The method of claim 1, wherein the metadata comprise scale information for the values.

18. The method of claim 1 further comprising a step of associating the signature-hash with the omics data set.

19. A method of comparing a plurality of omics data sets, comprising:
obtaining or generating a first signature-hash for a first omics data set, and obtaining or generating a second signature-hash for a second omics data set;
wherein each of the first and second signature-hashes comprise a plurality of values corresponding to allele frequencies for a plurality of SNPs in selected locations of the second omics data sets and further comprise metadata related to the selected locations; and
comparing the plurality of values for the first and second signature-hashes to determine a degree of relatedness.

20. The method of claim 19 wherein the first and second omics data sets have a format selected from the group of a SAM format, BAM format, and GAR format.

21. The method of any one of claims 19-20, wherein the selected locations are selected for at least one of SNP frequency, gender, ethnicity, and mutation type.

22. The method of any one of claims 19-21, wherein the values are based on a non-linear scale.

23. The method of any one of claims 19-22, wherein the values are expressed as hexadecimal values.

24. The method of any one of claims 19-23, wherein the first omics data set comprises the first signature-hash, and wherein the second omics data comprises the second signature-hash.

25. The method of any one of claims 19-24, wherein the degree of relatedness is based on SNP frequency, gender, ethnicity, and mutation type.

26. The method of any one of claims 19-25, wherein a predetermined degree of relatedness is indicative of common provenance.

27. The method of claim 19, wherein the selected locations are selected for at least one of SNP frequency, gender, ethnicity, and mutation type.

28. The method of claim 19, wherein the values are based on a non-linear scale.

29. The method of claim 19, wherein the values are expressed as hexadecimal values.

30. The method of claim 19, wherein the first omics data set comprises the first signature-hash, and wherein the second omics data comprises the second signature-hash.

31. The method of claim 19, wherein the degree of relatedness is based on SNP frequency, gender, ethnicity, and mutation type.

32. The method of claim 19, wherein a predetermined degree of relatedness is indicative of common provenance.

33. A method of identifying a single omics data set in a plurality of omics data sets having respective hashes, comprising:
obtaining or generating a single hash having a predetermined degree of relatedness to the single omics data set;
wherein each of the hashes comprises a plurality of values corresponding to allele frequencies for a plurality of SNPs in selected locations of an omics data set and further comprises metadata related to the selected locations;
comparing the plurality of values for the single hash with values of the hashes of each of the plurality of omics data sets; and
identifying the single omics data set in the plurality of omics data sets on the basis of a degree of relatedness between the values of the single hash and values of the hashes of each of the plurality of omics data sets.

34. The method of claim 33 wherein the single hash is obtained or generated from an additional omics data set.

35. The method of any one of claims 33-34, wherein the predetermined degree is identity of at least 90% of the plurality of values.

36. The method of any one of claims 33-35, wherein the predetermined degree is similarity of at least 90% of the plurality of values.

37. The method of any one of claims 33-36, wherein the selected locations are selected for at least one of SNP frequency, gender, ethnicity, and mutation type.

38. The method of any one of claims 33-37, further comprising a step of retrieving the single omics data set.

39. The method of any one of claims 33-38, wherein the step of comparing uses the metadata.

40. The method of claim 33, wherein the predetermined degree is identity of at least 90% of the plurality of values.

41. The method of claim 33, wherein the predetermined degree is similarity of at least 90% of the plurality of values.

42. The method of claim 33, wherein the selected locations are selected for at least one of SNP frequency, gender, ethnicity, and mutation type.

43. The method of claim 33, further comprising a step of retrieving the single omics data set.

44. The method of claim 33, wherein the step of comparing uses the metadata.

45. A method of identifying source contamination in an omics file, comprising:
providing a plurality of omics data sets having respective signature-hashes;
wherein each of the signature-hashes comprises a plurality of values corresponding to allele frequencies for a plurality of SNPs in selected locations of an omics data set and further comprises metadata related to the selected locations;
identifying at least some of the plurality of values of one of the omics data set in another omics data set.

46. The method of claim 45, wherein at least two of the plurality of omics data sets are from the same patient and are representative of at least two distinct points in time.

47. The method of any one of claims 45-46, wherein the selected locations are selected for at least one of SNP frequency, gender, ethnicity, and mutation type.

48. The method of any one of claims 45-47, wherein the step of identifying comprises a step of subtraction of corresponding values between at least two omics data sets.

49. The method of any one of claims 45-48, further comprising a step of identifying metadata in the one of the omics data set.

50. The method of claim 45, wherein the selected locations are selected for at least one of SNP frequency, gender, ethnicity, and mutation type.

51. The method of claim 45, wherein the step of identifying comprises a step of subtraction of corresponding values between at least two omics data sets.

52. The method of claim 45, further comprising a step of identifying metadata in the one of the omics data set.
